(19) United States
(12) Patent Application Publication
(10) Pub. No.: US 2019/0388672 A1
(43) Pub. Date: Dec. 26, 2019

(54) DRY-BOOSTER ADAPTER

(71) Applicant: ASP Global Manufacturing GmbH, Shaffhausen (CH)

(72) Inventors: Cole Gavin MCNEAL, Costa Mesa, CA (US); Samuel J. RHODES, Los Angeles, CA (US)

(21) Appl. No.: 16/446,858

(22) Filed: Jun. 20, 2019

Related U.S. Application Data
(60) Provisional application No. 62/687,685, filed on Jun. 20, 2018.

(51) Int. Cl. A61M 39/10; A61L 2/18; A61L 2/20; A61L 2/26

(52) U.S. Cl. CPC A61M 39/10; A61L 2/186; A61M 2039/1077; A61L 2/26; A61L 2202/24; A61L 2/208

(57) ABSTRACT

A dry-booster adapter assists in connecting a dry booster to an endoscope and may further assist in preventing detachment of the dry booster from the endoscope during a sterilization process. The adapter may include a fixation feature that fits between a surface of an endoscope control body and flanges thereof. It may also include another fixation feature including a locking tab. The adapter may also include a living hinge, which may assist in attaching the adapter to the endoscope, and that may be broken assist in removing the adapter from the endoscope.

The present application claims priority under 35 U.S.C. § 119 to U.S. Provisional Patent Application No. 62/687,685, filed Jun. 20, 2018. The entire contents of this application is incorporated by reference herein in its entirety.

### FIELD

The subject matter disclosed herein relates to dry boosters, which are devices that assist in sterilizing medical devices having lumens, e.g., endoscopes.

### BACKGROUND

Medical devices are typically sterilized before use in order to minimize the likelihood that a contaminated device might be used on a subject, which could cause an infection in the subject. Various sterilization techniques may be employed, such as steam, hydrogen peroxide, and vapor phase sterilization, either with or without a gas plasma and ethylene oxide (EtO).

Certain sterilization techniques are conducted at pressures other than ambient pressure or atmospheric pressure. For example the STERRAD® System, STERRAD® NX System or STERRAD® 100NX System of Advanced Sterilization Products, Division of Ethicon US, LLC, a Johnson & Johnson company, are examples of sterilization systems, or sterilizers, that vaporize hydrogen peroxide and operate at low pressures, e.g., less than 200 millitorr.

Various elongate medical devices having lumens, e.g., endoscopes, are challenging to sterilize for various reasons. For example, because pressure within a lumen decreases from the lumen's inlet as a function of length and diameter, the pressure drop must be overcome to ensure that sterilant passes through the entire lumen and reaches all surfaces of the lumen. Further, lumens may collect debris or be blocked by fluids, such as rinse water.

A dry booster is a device that may be connected to a lumen of an elongate medical device. When subject to a sterilization process in which pressure changes are implemented, pressure differentials between the inside of a dry booster at one end of the lumen and a pressure chamber at the other end of a lumen help pass a sterilant through the lumen, which assists in sterilizing the lumen.

### SUMMARY OF THE DISCLOSURE

A dry-booster adapter assists in connecting a dry booster to an endoscope and may further assist in preventing detachment of the dry booster from the endoscope during a sterilization process. A dry booster adapter is disclosed herein. The dry-booster adapter may include a body having a fixation feature. The fixation feature may have a thickness that is less than a distance between a surface of a flange of a port of a channel of an endoscope and a surface of a control head of the endoscope disposed opposite to the surface of the flange. Thus, for example, the distance between the surface of the flange and the surface of the control head may be between about 0.05 inches and 0.1 inches, whereas the thickness of the fixation feature may be between about 0.04 inches and 0.09 inches.

The adapter may also include a living hinge, which may be disposed proximate to the plate, e.g., on a surface the plate. A connector may be disposed through the plate. The connector may include a first end and a second end, the first end being insertable into the channel through the port, and the second end being matable to the dry booster. A spring may be disposed about the connector.

The dry booster adapter may also include a second fixation feature that includes a locking tab. The second fixation feature may also include a slot matable to the locking tab in one direction such that motion of the slot in a reverse direction is restricted. The slot may be disposed through the plate, which may also include the locking tab. The locking tab may comprise an elongate finger having a tooth matable to a surface adjacent to the slot.

The adapter may also include a second connector disposed through the plate that may be mated to and between the dry booster and a second channel of the endoscope through a second port such that the second connector and the second channel are in fluid communication.

The adapter may also include a pull tab on the plate and oriented transverse to the living hinge. It may also include a top surface in contact with the plate such that the locking tab may extend transversely from the top surface. The plate may further include a second living hinge, which may be oriented parallel to the first living hinge and spaced therefrom by a distance about equal to a thickness of the adapter's top surface.

Alternatively, the slot may be disposed through a first side surface of the adapter. In such embodiments, a connector body may extend from a second side surface and the connector body may include the second living hinge and the locking tab. The locking tab and the second side surface may be disposed on opposite sides of the connector body. Additionally, the first living hinge may connect the plate to the first side surface, while a third living hinge may connect the first side surface to the first fixation feature.

The dry-booster in any of the embodiments described above may be used to connect a dry booster and an endoscope, typically to assist in sterilizing the endoscope during a low pressure sterilization cycle. An exemplary method of use and variations thereof may include the following steps. First the endoscope and the dry-booster adapter may be received. The first end of the connector or connectors may be inserted into the port or ports of the endoscope's channel or channels. Then, the fixation feature (i.e., the first fixation feature where there are two fixation features) may be disposed between the flange and the control head. Then, the other end of the connector or connectors may be attached to the dry booster. Where the adapter includes two fixation features, the locking tab may be mated through the slot

Once the dry booster is connected to the endoscope by the dry-booster adapter, the endoscope may be sterilized using, e.g., a gaseous sterilant. Typically after sterilization, the dry-booster adapter may be removed from the endoscope. Such removal includes a step of breaking the living hinge or at least one of the living hinges, e.g., with the assistance of the pull tab. After the living hinge is broken, the connector or connectors may be removed from the ports and the adapter removed from the endoscope. Accordingly, the method may further comprise removing the connector(s) from the channel(s) and the adapter from the endoscope. Because the adapter has been broken, it is unsuitable for further use and may be discarded.

### BRIEF DESCRIPTION OF THE DRAWINGS

While the specification concludes with claims, which particularly point out and distinctly claim the subject matter described herein, it is believed the subject matter will be better understood from the following description of certain examples taken in conjunction with the accompanying drawings, in which like reference numerals identify the same elements and in which:

FIG. 1 depicts a first perspective view of a first embodiment of a dry booster adapter;

FIG. 2 depicts a second perspective view of the first embodiment of the dry booster adapter of FIG. 1;

FIG. 3 depicts a top view of a component of the dry booster adapter of FIG. 1;

FIGS. 4A-4E depict various steps of using the dry booster adapter of FIG. 1;

FIGS. 5A-5C depict various steps of using a second embodiment of the dry booster adapter; and

FIGS. 6A-6D depict various steps of using a third embodiment of the dry booster adapter.

### MODES OF CARRYING OUT THE INVENTION

The following detailed description should be read with reference to the drawings, in which like elements in different drawings are identically numbered. The drawings, which are not necessarily to scale, depict selected embodiments and are not intended to limit the scope of the invention. The detailed description illustrates by way of example, not by way of limitation, the principles of the invention. This description will clearly enable one skilled in the art to make and use the invention, and describes several embodiments, adaptations, variations, alternatives and uses of the invention, including what is presently believed to be the best mode of carrying out the invention.

As used herein, the terms “about” or “approximately” for any numerical values or ranges indicate a suitable dimensional tolerance that allows the part or collection of components to function for its intended purpose as described herein. More specifically, “about” or “approximately” may refer to the range of values ±10% of the recited value, e.g. “about 90%” may refer to the range of values from 81% to 99%. In addition, as used herein, the terms “patient,” “host,” “user,” and “subject” refer to any human or animal subject and are not intended to limit the systems or methods to human use, although use of the subject invention in a human patient represents a preferred embodiment.

Dry boosters may be used to help draw chemical sterilants into a lumen or lumens of an elongate medical device, e.g., an endoscope. Dry boosters are disclosed in U.S. Pat. Nos. 6,451,255 and 7,229,591, which are hereby incorporated by reference in their entirety. A dry booster may include an adapter to assist in establishing a connection between the dry booster and a lumen of an elongate medical device. An exemplary adapter is described in U.S. Pat. No. 6,187,265, which is hereby incorporated by reference in its entirety.

During many sterilization procedures involving a chemical sterilant, such as hydrogen peroxide, pressure within the sterilizer's vacuum chamber may be decreased or increased considerably and/or quickly. For example, the pressure in the vacuum chamber may be decreased from approximately 760 torr to approximately 2 torr over a time ranging between approximately 30 seconds to 15 minutes. For example, in the STERRAD® 100NX System, various phases of a sterilization cycle are performed during which pressure is lowered from approximately 760 torr to approximately 2 torr at differing rates based on the purpose of the phase. For example, some phases include injecting a sterilant, e.g., a gaseous sterilant such as hydrogen peroxide (which slows down the rate of pressure change), whereas other phases are performed to remove residual moisture from within the chamber (which may cause evaporation that can also slow down the rate of pressure change), whereas other phases do not include injecting a sterilant or removing residual moisture (which may correspond to the fastest pressure change). The STERRAD® 100NX phase that has the slowest change in pressure may take from approximately 5 minutes to approximately 8 minutes to lower the pressure from approximately 760 torr to approximately 2 torr. The STERRAD® 100NX phase that has the fastest change in pressure takes approximately 1.5 minutes to change the pressure from approximately 760 torr to approximately 2 torr. Thus the average rate of negative change of pressure in the STERRAD® 100NX System's vacuum chamber may be between approximately 90 torr per minute and 510 torr per minute. Pressure changes over a portion of a cycle or instantaneous pressure changes, dP/dt, may be greater or less than the aforementioned average rates. Furthermore, the phases typically include an evacuation step during which the vacuum chamber is vented to the atmosphere and pressure within the vacuum chamber returns to atmospheric pressure. This pressure change may occur rapidly. For example, the pressure may be changed from approximately 150 mTorr to approximately 760 torr over a time ranging between approximately 10 seconds and one minute, such as thirty seconds. Thus, the pressure can be changed at a positive rate of approximately 1500 torr per minute.

In some instances, these pressure changes could cause a leak to occur between a dry booster and an endoscope to which it is attached. Such a leak or detachment may prevent, or lower the ability of, the dry booster to pull sterilant through the lumen, which would reduce the effectiveness of the sterilization process and raise the likelihood that the sterilization process would not sterilize the entire lumen.

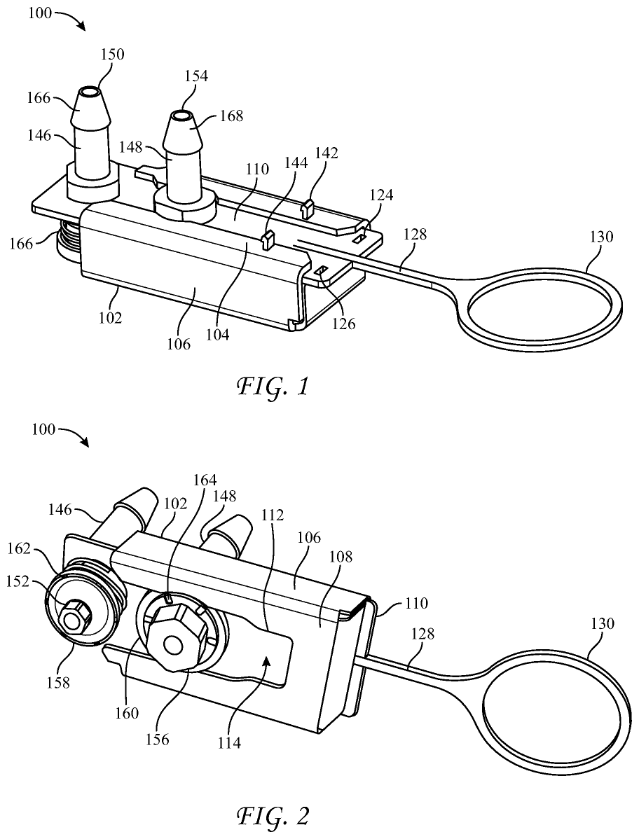

Disclosed herein is an adapter that may be used to improve the security of an attachment between a dry booster and an endoscope. A first embodiment of the adapter is reflected in FIGS. 1 and 2. Adapter 100 includes a body 102 having a top surface 104, a side surface 106, and a bottom surface 108. A channel 114 may further be disposed through bottom surface 108. An edge 112 of a portion of channel 114 may be considered a fixation feature because, as will be detailed below, edge 112 helps secure adapter 100 to the endoscope's control head.

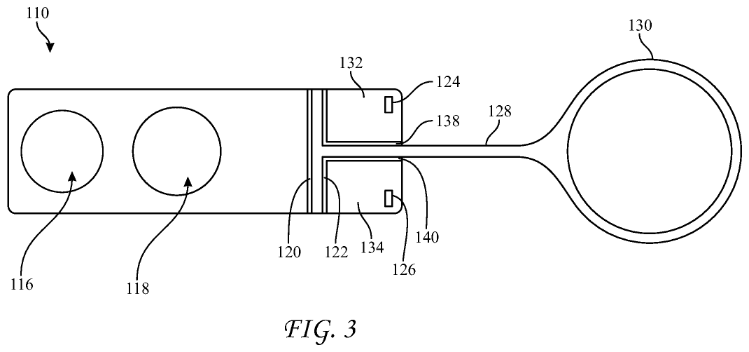

With further reference to FIG. 3, adapter 100 also includes a plate 110 that couples to body 102 such that it contacts an underside of top surface 104 and is slidable relative to body 102. Plate 110 includes at least one bore, e.g., bore 116 and bore 118, at least one living hinge, e.g., living hinge 120 and living hinge 122, at least one slot, e.g., slot 124 and slot 126, and a pull tab 128 that may optionally include a loop 130. Plate 110 may also include two corner portions 132 and 134 through which slots 124 and 126 may be respectively disposed. The boundaries of corner portions 132 and 134 may be edges of plate 110, living hinge 122 and pull tab 128. Further, a strip 138 of plate 110 disposed between corner portion 134 and pull tab 128 may be thinner than the portions of plate 110 around it, such that it may be torn to separate corner portion 134 from pull tab 128. Similarly, a strip 140 of plate 110 disposed between corner portion 136 and pull tab 128 may be thinner than the portions of plate 110 around it, such that it may be torn to separate corner portion 136 from pull tab 128.

Body 102 may further include at least one locking tab, e.g., locking tab 142 and locking tab 144, which may be mated through slots 124 and 126, respectively. Thus, the locking tabs and slots may together be considered a fixation feature whereby the slot is matable to the locking tab in one direction such that motion of the slot in a reverse direction is restricted. For example, corner portions 132 and 134 may be separated from pull tab 128 by tearing strips 138 and 140. Then, plate 110 may be bent or folded at living hinge 120, and, if included 122, to cause corner portions 132 and 134 to rotate about the living hinge such that they ultimately contact top surface 104. In this configuration, locking tabs 142 and 144 pass through slots 124 and 126 to mate thereto and secure corner portions 138 and 140 to top surface 104, which prevents plate 110 from sliding relative to body 102.

Dry-booster adapter 100 may further include at least one connector, e.g., connector 146 and connector 148. Both connectors may be connected to channels of an endoscope and to a dry booster to enable fluid communication therebetween. Dry-booster adapter 100 (and the dry boosters of the other exemplary embodiments disclosed herein), secure the connectors to the endoscope body to assist in preventing leaks at an interface between the connectors and the endoscope. Thus, connector 146 may have a first end 152 and a second end 150. Similarly, connector 148 may have a first end 156 and a second end 154. Flanges, e.g., flange 158 and flange 160, may additionally be included proximate to second ends 152 and 156 to assist with securing their respective connectors to the endoscope, as will be detailed below. Preferably, flanges 158 and 160 are made from a material that does not readily absorb gaseous sterilants, such as hydrogen peroxide, to avoid increasing the sterilization challenge at a junction between the flanges and ports of the endoscope channels. For example, the flanges may be fabricated from high-density polyethylene or a silicone (preferably high durometer). Further, they may include bumps 162 and/or grooves 164 to further assist the sterilant in reaching all surfaces.

Connectors 146 and 148 may have a form akin to tubing connectors, e.g., with barb connectors 166 and 168 disposed on the respective second ends 150 and 154. Second ends 150 and 154 may be connected to a dry booster directly or indirectly, e.g., via tubing.

Connector 146 may be disposed through bore 116 and connector 148 may be disposed through bore 118. Accordingly, connectors 146 and 148 may move relative to body 102 along with plate 110, or be fixed relative to body 102 along with plate 110, depending on whether corner portions 132 and 134 have been secured to top surface 104 via locking tabs 142 and 144 and slots 124 and 126.

Connectors 146 and 148 may be tubular in structure, having a cylindrical wall. Springs may be disposed around the connectors and be disposed between plate 110 and flanges 158, 160. In FIGS. 1 and 2, spring 166 about connector 146 is visible, but spring 168 about connector 148 is hidden. The springs press flanges 158 and 160 against ports on the endoscope control head, which helps maintain a seal between the connectors and the channels of the endoscope.

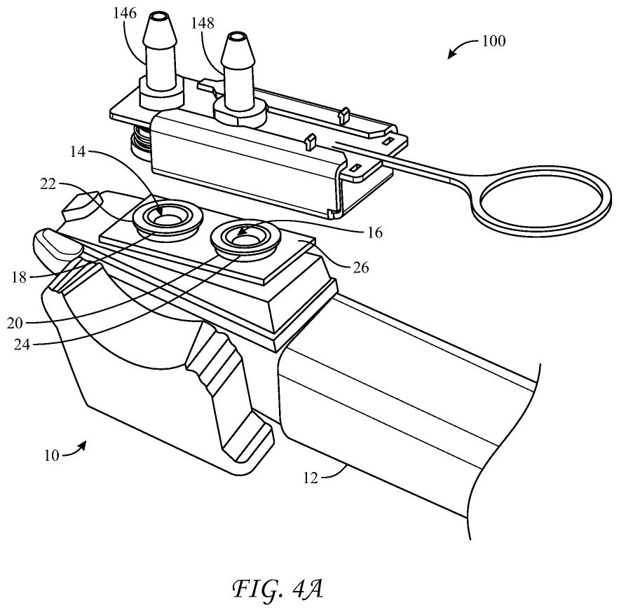

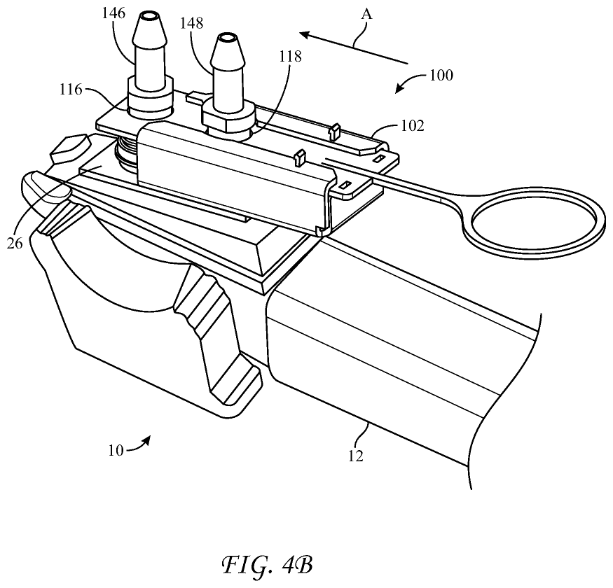

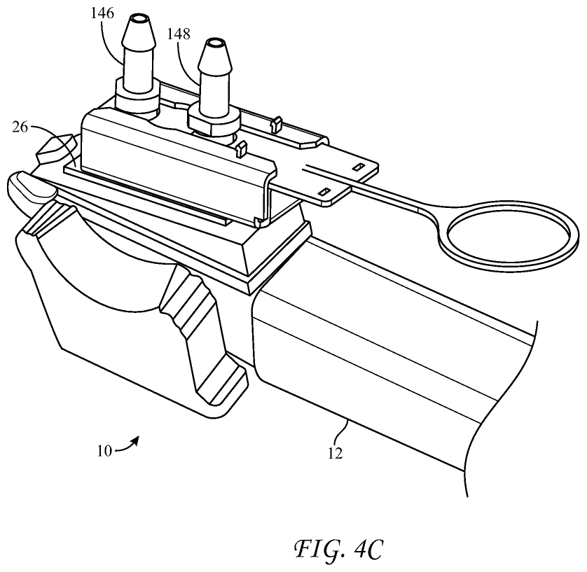

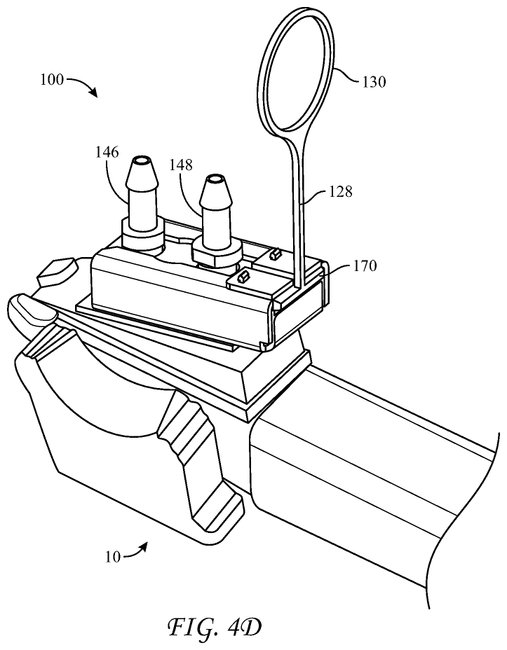

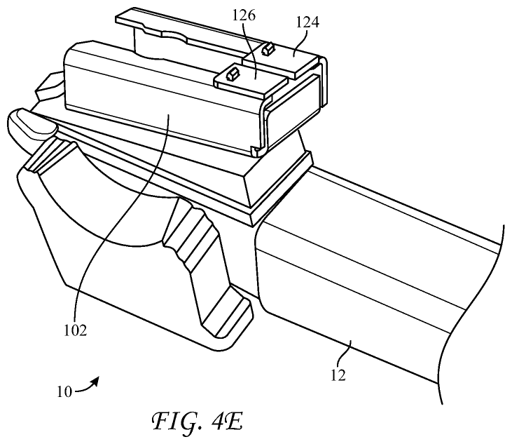

FIGS. 4A-4E reflect various steps of using dry-booster adapter 100 with an endoscope 10, including securing it thereto and removing it therefrom. Endoscope 10 includes a control head 12, which provides access to two of the endoscope's channels 14 and 16 (e.g., the suction channel and air/water channel) via ports 18 and 20. Ports 18 and 20 may each further include flanges, i.e., flange 22 on port 18 and flange 24 on port 22. Flanges 22 and 24 may be disposed above a control-head surface 26 by between approximately 0.05 inches and 0.1 inches, e.g., between approximately 0.07 and 0.08 inches.

As shown in FIG. 4A, connector 146 is aligned with channel 14 and connector 148 is aligned with port 16. First ends 152 and 156 are then positioned inside of channels 14 and 16 and bottom surface 108 of body 102 is brought into contact with surface 26 of control head 12. As seen in FIG. 4B, this causes connectors 146 and 148 to be moved upward through bores 116 and 118. Accordingly, springs 146 and 148 become compressed and thus apply a compression force between flanges 158 and 160 on the one hand and ports 18 and 20 on the other, which assists in maintaining second ends 152 and 156 in their respective channels and also restrains plate 110 relative thereto. Body 102 may then be slid forward, i.e., in the direction of arrow A, along plate 110, to the position reflected in FIG. 4C. This movement results in sliding channel 114, including edge or fixation feature 112, between flanges 158 and 160 on one side and surface 26 on the other. Thus, in this configuration, connectors 146 and 148 may be restrained in a direction parallel to their longitudinal axes. To assist in providing a secure restraint that may be readily slid and disposed between surface 26 and flanges 158, 160, the thickness of bottom surface 108, and thus edge or fixation feature 112, should be close to but less than the distance between surface 26 and flanges 158, 160. Based on the exemplary dimensions provided above, the thickness of fixation feature 112 may be between about 0.04 inches and 0.09 inches, e.g., about 0.06 inches.

Dry-booster adapter 100 may next be locked in place by restraining further movement of body 102 relative to control head 12 and connectors 148 and 148, i.e., in a direction opposite to Arrow A in FIG. 4B. First, corner portions 132 and 134 may be separated from pull tab 128 by tearing strips 138 and 140. Second, corner portions 132 and 140 may be rotated about living hinges 120 and 122 to bring them into contact with top surface 104 of body 102 such that locking tabs 142 and 144 are inserted through slots 124 and 126. In this configuration, adapter 100 may be connected to a dry booster, either directly or indirectly, e.g., via tubing. Then, the entire assemblage, i.e., endoscope 10 connected to the dry booster via adapter 100, may be placed into a sterilization chamber of a sterilizer and sterilized, e.g., with a gaseous sterilant, such as hydrogen peroxide.

FIG. 4E reflects a step during the removal of adapter 100 from endoscope 10. Typically, such removal is performed following sterilization when the endoscope is returned to a sterile field of an operating room for use in the next procedure. First, pull tab 128 is separated from the remainder of plate 100, including corner portions 124 and 126. The user may pull on tab 128 or on loop 130, which causes living hinges 120 and 122 to tear on both sides of tear portion 170 (FIG. 4D) and disconnect corner portions 124 and 126 from the rest of plate 110 such that body 102 may be again moved relative to plate 110. By moving body 102 in a direction opposite to the direction of arrow A (FIG. 4B), i.e., the direction of attachment, or by moving plate 110 in the direction of arrow A, adapter 100 may be removed from endoscope 10. As seen in FIG. 4E, tab 128 and tear portion have been removed, connectors 146 and 148 have been removed, but body 102 and corner portions 124 and 126, still connected thereto, have yet to be removed. Further, because the adapter has been broken by tearing living hinges 120 and 122, it is unsuitable for further use and may be discarded.

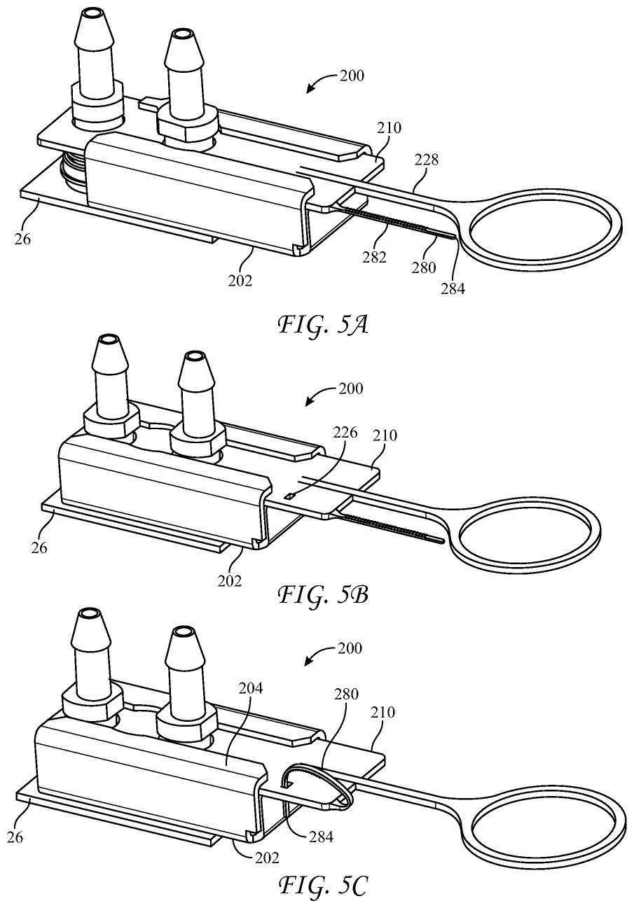

Another embodiment is reflected in FIGS. 5A-5C, showing dry-booster adapter 200 in various stages of use and attachment to control-head surface 26 and the ports thereon. Adapter 200 functions in the same manner as, and includes similar components to adapter 100. The differences between the two embodiments concerns the fixation feature that restricts relative movement between body 202 and plate 210. Thus, instead of locking tabs 142 and 144, adapter 200 includes at least one finger, e.g., finger 280. Finger 280 may include thereon at least one tooth 282. Finger 280 is flexible and may be deformed to insert tip 284 and a portion of finger 280 through slot 226. Tooth 282 restricts removal of finger 280 from slot 226. As shown in FIG. 5C, the space present between finger 280 and top surface 204 of body 202 is exaggerated. Finger 5C blocks relative motion between body 202 and plate 210. Other than this, the structure of adapter 200 may be identical to the structure of adapter 100. Further it is used in largely the same fashion, including connection to control-head surface 26, connection to a dry booster, and using pull tab 228 to tear the living hinges.

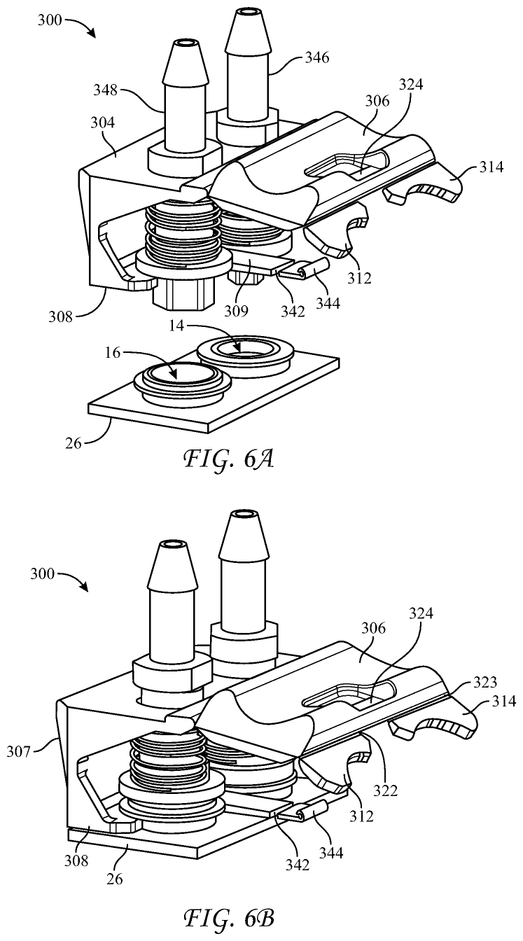

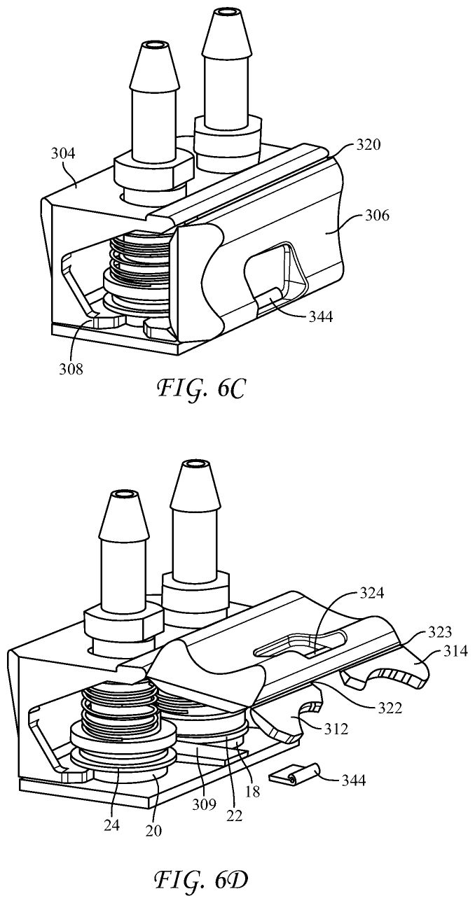

Another embodiment is reflected in FIGS. 6A-6D, showing dry-booster adapter 300 in various stages of use and attachment to control-head surface 26 and the ports thereon. Adapter 300 includes a body 302 having a top surface 304, a first side surface 306 including a slot or slit 324, a second side surface 307, a bottom surface 308, and bottom portions 312 and 314. Living hinge 320 may be included between top surface 304 and first side surface 306. Living hinge 322 may be included between first side surface 306 and first bottom portion 312. Living hinge 323 may be included between first side surface 306 and first bottom portion 314. A connector portion or connector body 309 extends from second side surface 307. A locking tab or fixation feature 344 may be disposed on the end of connector body 309 opposite from second side surface 307. Further, a living hinge 342 may be disposed between second side surface 307 and locking tab 344.

In use adapter 300 is first aligned with control-head surface 26 by aligning connector 346 with channel 14 and connector 348 with channel 16, as in FIG. 6A. Then, the tips of these connectors are inserted into the respective channels, as in FIG. 6B, while bottom surface 308 is positioned between control-head surface 26 and the flanges 22 and 24 of the ports of channels 14 and 16, which also positions connector body 309 underneath flanges 22 and 24, between ports 18 and 20. Next, first side surface 306 is rotated about living hinge 320 and bottom portions 312 and 314 are rotated respectively about living hinges 322 and 323, such that bottom portions 312 and 314 may be positioned between control-head surface 26 and flanges 22 and 24, and locking tab 344 passes through slit 324, as seen in FIG. 6C. In this configuration, adapter 300 is locked onto control-head surface 26. Removal of adapter 300 from control-head surface 26 is effected by pulling on first surface 306, which causes living hinge 342 to tear such that tab 344 becomes separated from connector body 309, as seen in FIG. 6D. This allows side surface 306 and bottom portions 312 and 314 to rotate, permitting adapter 300 to be removed. Further because the adapter has been broken at living hinge 342, it is unsuitable for further use and may be discarded.

Any of the examples or embodiments described herein may include various other features in addition to or in lieu of those described above. The teachings, expressions, embodiments, examples, etc., described herein should not be viewed in isolation relative to each other. Various suitable ways in which the teachings herein may be combined should be clear to those skilled in the art in view of the teachings herein.

Having shown and described exemplary embodiments of the subject matter contained herein, further adaptations of the methods and systems described herein may be accomplished by appropriate modifications without departing from the scope of the claims. In addition, where methods and steps described above indicate certain events occurring in certain order, it is intended that certain steps do not have to be performed in the order described but in any order as long as the steps allow the embodiments to function for their intended purposes. Therefore, to the extent there are variations of the invention, which are within the spirit of the disclosure or equivalent to the inventions found in the claims, it is the intent that this patent will cover those variations as well. Some such modifications should be apparent to those skilled in the art. For instance, the examples, embodiments, geometrics, materials, dimensions, ratios, steps, and the like discussed above are illustrative. Accordingly, the claims should not be limited to the specific details of structure and operation set forth in the written description and drawings.

We claim:

1. A dry-booster adapter, comprising:
a body including a fixation feature having a thickness that is less than a distance between a surface of a flange of a port of a channel of an endoscope and a surface of a control head of the endoscope disposed opposite to the surface of the flange;
a plate;
a living hinge disposed proximate to the plate, and
a connector disposed through the plate, the connector including a first end and a second end, the first end being insertable into the channel through the port, and the second end being matable to the dry booster.

2. The dry booster adapter of claim 1, wherein the living hinge is disposed on the plate.

3. The dry booster adapter of claim 1, wherein the fixation feature is a first fixation feature; and
the adapter further comprises a second fixation feature including a locking tab.

4. The dry booster adapter of claim 3 wherein the second fixation feature further includes a slot matable to the locking tab in one direction such that motion of the slot in a reverse direction is restricted.

5. The dry booster adapter of claim 3, further comprising a spring disposed about the connector.

6. The dry booster adapter of claim 3, wherein the connector is a first connector, the channel is a first channel, and the port is a first port; and
the adapter further comprises a second connector disposed through the plate, the second connector including a first end and a second end, the first end being matable to a second channel of the endoscope through a second port such that the second connector and the second channel are in fluid communication, and the second end of the second connector being matable to the dry booster.

7. The dry booster adapter of claim 4, wherein the plate includes a pull tab oriented transverse to the living hinge.

8. The dry booster adapter of claim 7 wherein the slot is disposed through the plate, the plate includes the locking tab, and the locking tab comprises an elongate finger having a tooth matable to a surface adjacent to the slot.

9. The dry booster adapter claim 7, further comprising a top surface in contact with the plate, the locking tab extending transversely from the top surface, and wherein the living hinge is a first living hinge and the plate further includes a second living hinge, the second living hinge being oriented parallel to the first living hinge and spaced therefrom by a distance about equal to a thickness of the top surface.

10. The dry booster adapter of claim 4, wherein the living hinge is a first living hinge and the adapter further comprises:
a first side surface through which the slot is disposed;
a second side surface from which a connector body extends, the connector body including a second living hinge and the locking tab.

11. The dry booster adapter of claim 10, wherein the locking tab and the second side surface are disposed on opposite sides of the connector body.

12. The dry booster adapter of claim 11, wherein the first living hinge connects the plate to the first side surface.

13. The dry booster adapter of claim 12, further comprising a third living hinge that connects the first side surface to the first fixation feature.

14. A method of using a dry-booster adapter, comprising:
receiving an endoscope comprising
a channel, and
a control head including a port disposed about an opening of the channel, the port having a flange;
receiving a dry-booster adapter comprising
a body including a fixation feature having a thickness that is less than a distance between a surface of the flange and a surface of the control head disposed opposite to the surface of the flange,
a plate,
a living hinge disposed proximate to the plate, and
a connector disposed through the plate, the connector including a first end and a second end, the first end being insertable into the channel through the port, and the second end being matable to the dry booster;
inserting the first end of the connector into the channel through the port;
disposing the fixation feature between the flange and the control head; and
attaching the second end of the connector to a dry booster.

15. The method of claim 14, further comprising sterilizing the endoscope using a gaseous sterilant.

16. The method of claim 15, further comprising breaking the living hinge.

17. The method of claim 16, further comprising removing the first end of the connector from the channel.

18. The method of claim 16, wherein the fixation feature is a first fixation feature, and the adapter further comprises a second fixation feature including a locking tab, and a slot matable to the locking tab in one direction such that motion of the slot in a reverse direction is restricted.

19. The method of claim 18, further comprising mating the locking tab through the slot before the step of sterilizing the endoscope.
